(19) United States
(12) Patent Application Publication
(10) Pub. No.: US 2023/0326585 A1
(43) Pub. Date: Oct. 12, 2023

(54) SYSTEM AND METHOD TO FACILITATE ADHERENCE TO MULTIPLE MEDICAL CARE PLANS

(71) Applicant: Adherelive Private Limited, Delhi (IN)

(72) Inventors: Vivek Asthana, Meerut (IN); Ankur Agrawal, Gurugram (IN)

(21) Appl. No.: 18/043,160

(22) PCT Filed: Aug. 27, 2021
(86) PCT No.: PCT/IB2021/057851

§ 371 Date: Feb. 27, 2023

(30) Foreign Application Priority Data
Aug. 28, 2020 (IN) 202041037117

(51) Int. Cl. G16H 40/20; G16H 10/60; G16H 20/10; G16H 80/00; G16H 15/00

(52) U.S. Cl. CPC G16H 40/20; G16H 10/60; G16H 20/10; G16H 80/00; G16H 15/00

(57) ABSTRACT

A system to facilitate adherence to multiple medical care plans is disclosed. The system includes a consent management module, configured to classify the information received from the biophysical data capturing module and the health status recording module in accordance with predesignated privacy matrix level and configured to provide access to a specific privacy level of classified information associated with a specific disease to one of one or more doctors, upon receiving consent from at least one of one or more registered doctors, the registered patient and the one or more registered healthcare personnel. The system includes a graphic user interface module, configured to provide integrated view of the information, configured to enable the one or more registered doctors to collaboratively intervene according to information provided and also configured to enforce adherence of the registered patient to the one or more treatment plans.

### EARLIEST PRIORITY DATE

This application claims priority from a complete patent application filed in India having Patent Application No. 202041037117, filed on Aug. 28, 2020, and titled “SYSTEM AND METHOD TO FACILITATE ADHERENCE TO MULTIPLE MEDICAL CARE PLANS” and claims priority from a PCT patent application having Patent Application No. PCT/IB2021/057851, filed on Aug. 27, 2021, and titled “SYSTEM AND METHOD TO FACILITATE ADHERENCE TO MULTIPLE MEDICAL CARE PLANS”.

### FIELD OF INVENTION

Embodiments of a present disclosure relates to field of healthcare, and more particularly to a system and a method to facilitate adherence to multiple medical care plans.

### BACKGROUND

A critical health problem may interrupt all aspects of one's life. Here, the health problem may be recurring or life-threatening, such as cancer, or a major health event such as a stroke, heart attack, or a simple debilitating injury. It is important to understand that an individual may develop serious health problems unexpectedly or without warning.

A patient, at any instance may suffer from multiple health problems, and it is vital for the patient to consult specialized doctors for each problem. Conventionally, no system enables proper interaction facility for multiple doctors addressing multiple problems in a single patient. Here, the patient must manually disclose to a specific doctor about other diseases or ongoing disease specific treatment plans.

Such manual disclosure often leads to confusion and uncertainty. The patient medical history should be available to each consulting doctor for generating a better treatment plan and identifying possible scenarios of unexpected medicine reactions/results. Micro-health interactions between patients and multiple doctors is important for managing treatments for avoidance of preventable medical conditions/compilations or hospital admissions.

Privacy issues plays very critical role while multiple doctors collaborate to treat multiple diseases of a single patient. Since medical data is digitised, in most of the cases it becomes so difficult to ensure privacy or obtain consent from patients and doctors in real time without inflicting delay in treatments.

Furthermore, each biological system corresponding to our body are related to each other in some or the other way. So basically, diseases in a patient may be better tackled if multiple specialized doctors work in collaboration. In such collaboration, chances for improvement of the patient's health also increases.

Hence, there is a need for an improved system to facilitate adherence to multiple medical care plans and a method to operate the same and therefore address the aforementioned issues.

### BRIEF DESCRIPTION

In accordance with one embodiment of the disclosure, a system to facilitate adherence to multiple medical care plans is disclosed. The system includes one or more processors hosted on a server. The system also includes an entity registration module operable by the one or more processors. The entity registration module is configured to register one or more entities with a plurality of information.

The system also includes a biophysical data capturing module operable by the one or more processors. The biophysical data capturing module is operatively coupled to the entry registration module. The biophysical data capturing module is configured to capture information regarding at least one of one or more diseases, one or more bio-physical parameters, type of discomfort, severity of discomfort, part of body experiencing the discomfort and time of discomfort, associated with the registered patient.

The system also includes a health status recording module operable by the one or more processors. The health status recording module is operatively coupled to the entity registration module. The health status recording module is configured to record medical information, in a consolidated database representative of one or more symptoms, one or more medication side-effects, ongoing medication, one or more treatment plans, and severity of health condition via communicatively coupled one or more devices associated with at least one of the registered patient, one or more registered healthcare personnel and one or more registered doctors.

The system also includes a consent management module operable by one or more processors. The consent management module is operatively coupled to the to the health status recording module and the biophysical data capturing module. The consent management module is configured to classify the information received from the biophysical data capturing module and the health status recording module in accordance with predesignated privacy matrix level. The consent management module is configured provide access to a specific privacy level of classified information associated with a specific disease to one of one or more doctors, upon receiving consent from at least one of one or more registered doctors, the registered patient and the one or more registered healthcare personnel.

The system also includes a graphic user interface module operable by one or more processors. The graphic user interface module is operatively coupled to the to the biophysical data capturing module, the health status recording module, and the consent management module. The graphic user interface module is configured to provide integrated view of the information received from the biophysical data capturing module and the health status recording module to the one or more registered doctors treating the registered patient for one or more diseases based on the consent specific to privacy level of the classified information. The graphic user interface module is also configured to enable the one or more registered doctors to collaboratively intervene according to information provided, wherein the graphic user interface module flags conflict during the collaboratively intervention by more than two doctors. The graphic user interface module is also configured to enforce adherence of the registered patient to the one or more treatment plans by processing the information received from the biophysical data capturing module and the health status recording module.

In accordance with one embodiment of the disclosure, a method for facilitating adherence to multiple medical care plans is disclosed. The method includes registering one or more entities with a plurality of information. The method also includes capturing. information regarding at least one of one or more diseases, one or more bio-physical parameters, type of discomfort, severity of discomfort, part of body experiencing the discomfort and time of discomfort, associated with the patient The method also includes recording medical information representative of one or more symptoms, one or more medication side-effects, ongoing medication, one or more treatment plans, and severity of health condition via communicatively coupled one or more devices associated with at least one of the registered patient, one or more registered healthcare personnel and one or more registered doctors.

The method also includes classifying the information received from the biophysical data capturing module and the health status recording module in accordance with predesignated privacy matrix level. The method also includes providing access to a specific privacy level of classified information associated with a specific disease to one of one or more registered doctors, upon receiving consent from at least one of one or more registered doctors, the registered patient and the one or more registered healthcare personnel.

The method also includes providing integrated view of the information received from the biophysical data capturing module and the health status recording module to the one or more registered doctors treating the registered patient for one or more diseases based on the consent specific to privacy level of the classified information. The method also includes enabling the one or more registered doctors to collaboratively intervene according to information provided. The method also includes flagging conflict during the collaboratively intervention by more than two doctors. The method also includes enforcing adherence of the registered patient to the one or more treatment plans by processing the information received from the biophysical data capturing module and the health status recording module.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a system to facilitate adherence to multiple medical care plans in accordance with an embodiment of the present disclosure;

FIG. 2 is a schematic representation of an embodiment representing input mechanism window related to disease corresponding to a registered patient of FIG. 1 in accordance with an embodiment of the present disclosure;

FIG. 3 is a schematic representation of an embodiment representing the integrated presentation window corresponding to the registered patient of FIG. 1 in accordance with an embodiment of the present disclosure;

FIG. 4 is a schematic representation of an embodiment representing the adherence dashboard window corresponding to the registered patient of FIG. 1 in accordance with an embodiment of the present disclosure;

FIG. 5 is a schematic representation of an embodiment representing the adherence dashboard window 120 with real time information corresponding to the registered patient of FIG. 1 in accordance with an embodiment of the present disclosure;

FIG. 6 is a schematic representation of an embodiment representing the system to facilitate adherence to multiple medical care plans of FIG. 1 in accordance with an embodiment of the present disclosure;

FIG. 7 is a block diagram of a computer or a server in accordance with an embodiment of the present disclosure;

FIG. 8 is a flowchart representing the steps of a method 200 to facilitate adherence to multiple medical care plans in accordance with an embodiment of the present disclosure; and

FIG. 9 is a flowchart representing the steps of a method 260 to facilitate adherence to multiple medical care plans in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to system and method to facilitate adherence to multiple medical care plans. The system incudes various dedicated modules configured to register one or more entities into the system based on a plurality of information, capture information regarding at least one of one or more diseases, one or more bio-physical parameters, type of discomfort, severity of discomfort, part of body experiencing the discomfort and time of discomfort, associated with the registered patient, record medical information associated with at least one of the registered patient, one or more registered healthcare personnel and one or more registered doctors, classify the information received from the biophysical data capturing module and the health status recording module in accordance with predesignated privacy matrix level, provide access to a specific privacy level of classified information associated with a specific disease to one of one or more doctors, upon receiving consent from at least one of one or more registered doctors, the registered patient and the one or more registered healthcare personnel, provide integrated view of the information received from the biophysical data capturing module and the health status recording module to the one or more registered doctors treating the registered patient for one or more diseases based on the consent specific to privacy level of the classified information, enable the one or more registered doctors to collaboratively intervene according to information provided, and enforce adherence of the registered patient to the one or more treatment plans by processing the information received from the biophysical data capturing module and the health status recording module.

A computer system (standalone, client or server computer system) configured by an application may constitute a “platform” or “module’ that is configured and operated to perform certain operations. In one embodiment, the “platform” or “module’ may be implemented mechanically or electronically, so a platform or module may comprise dedicated circuitry or logic that is permanently configured (within a special-purpose processor) to perform certain operations. In another embodiment, a “platform” or “module” may also comprise programmable logic or circuitry (as encompassed within a general-purpose processor or other programmable processor) that is temporarily configured by software to perform certain operations.

Accordingly, the term “module’ or “platform’ should be understood to encompass a tangible entity, be that an entity that is physically constructed permanently configured (hardwired) or temporarily configured (programmed) to operate in a certain manner and/or to perform certain operations described herein.

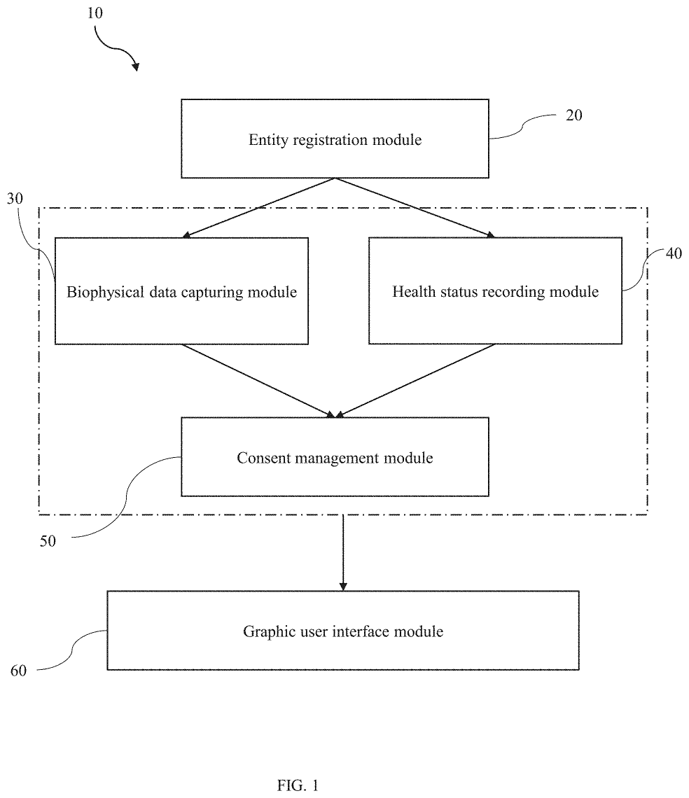

FIG. 1 is a block diagram representation of a system 10 to facilitate adherence to multiple medical care plans in accordance with an embodiment of the present disclosure. Adherence to a medication regimen is a vitally important element to regimen efficacy and a desired therapeutic outcome. A patient, in one instance may confer to multiple specialised doctors for different illnesses. It is important that the patient gets proper guidance about conflict of different medication regimes. Moreover, managing consent between multiple doctors as well as patients is also an essential element for better health care system which respects the privacy of entities without compromising on efficacy of the system. In one embodiment, the system 10 is hosted on a Software as a service (SaaS) platform.

The system 10 includes an entity registration module 20 operable by the one or more processors. The entity registration module 20 is configured to register one or more entities with a plurality of information. In one embodiment, the one or more entities comprises a patient, one or more healthcare personnel, one or more doctors, an administrator and a patient contact. In such embodiment, each of the one or more entities will have to provide specific information for registration.

In one specific embodiment, the patient for registration may provide information such as name, age, contact address, present location, past medical history and the like. In another specific embodiment, the one or more doctors for registration may provide information such as name, contact address, area of specialization, hospital or clinic they are associated with, timings of visit and the like. In such embodiment, the registered patient may be undergoing treatment for one or more diseases by the one or more registered doctors.

Similarly, the one or more healthcare personnel for registration may provide information such as name, contact details, area of specialization, location and the like. Similarly, the one or more registered healthcare personnel with different specialization may be a caregiver to a single registered patient. The one or more healthcare personnel may include allied healthcare personnel, geriatric care personnel and the like. As used herein, the term “administrator” refers to a person responsible for carrying out the administration of the system 10. Here, patient contact refers to a person responsible with health liability of registered patient.

The system 10 also includes a biophysical data capturing module 30 operable by the one or more processors. The biophysical data capturing module 30 is operatively coupled to the entry registration module 20. The biophysical data capturing module 30 is configured to capture information regarding at least one of one or more diseases, one or more bio-physical parameters, type of discomfort, severity of discomfort, part of body experiencing the discomfort and time of discomfort, associated with the registered patient.

In one embodiment, the patient provides the biophysical data via at least one of textual input, pre-designated field input and input on 3D image representative of human body. In another embodiment, real-time information regarding the one or more diseases and the one or more bio-physical parameters of the registered patient is captured via IOT enabled one or more wearable sensors.

As used herein, the term “disease” refers to a disorder of structure or function in a human, especially one that produces specific symptoms or that affects a specific location and is not simply a direct result of physical injury. The system 10 via the biophysical data capturing module 30 captures information about specific disease affecting the registered patient body along with additional medical details. Discomfort may be the ongoing pain or uneasiness caused by the recorded disease.

In one specific embodiment, the registered patient may provide all medical information as stated via textual input. In such embodiment, the biophysical data capturing module 20 uses data extraction techniques to look deeper into provided textual inputs to classify and extract relevant information in a structured manner. After data extraction, the input information is interpreted for understanding the disease or discomfort related to the recorded disease.

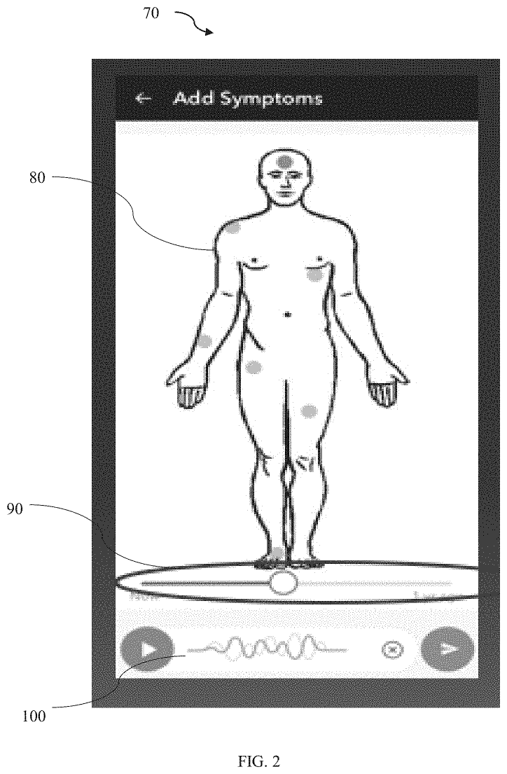

In another specific embodiment, an easy approach would be to provide an input via 3D image representative of human body. FIG. 2 is a schematic representation of an embodiment representing input mechanism window 70 related to disease corresponding to a registered patient of FIG. 1 in accordance with an embodiment of the present disclosure. A text or voice input bar 100 is located at the bottom end of the recording input window 70. It is pertinent to note that, the registered patient may also provide voice input as well as pictorial input via the text or voice input bar 100. In specific embodiment, the patient may give input by touching on screen of smart device or clicking with pointing device a specific body part or parts on the 3D image representative of the body, where the 3d image body 80 may be zoomed in or out and 360° rotated by a slider and arrows buttons 90. In another embodiment, all such details provided by the registered patient may be showcased on the 3D image body 80 as shown in FIG. 2.

In yet another embodiment, the IoT enabled one or more wearable sensors include smart watch, smart bangle, patch, wearable clothes and the like. In such embodiment, any bio-physical parameter such as, but not limited to, heart rate, body pressure level may be easily be recorded in real time for better tracking.

The system 10 also includes a health status recording module operable 40 by the one or more processors. The health status recording module 40 is operatively coupled to the entity registration module 20. The health status recording module 40 is configured to record medical information, in a consolidated database representative of one or more symptoms, one or more medication side-effects, ongoing medication, one or more treatment plans, and severity of health condition via communicatively coupled one or more devices associated with at least one of the registered patient, one or more registered healthcare personnel and one or more registered doctors.

Here, the one or more devices may be IoT enabled one or more wearable sensor devices, handheld devices or any linked medical device. It is pertinent to note that the system 10 via real time notification realizes about one or more symptoms and one or more medication side-effects. For example, the system 10 may generate “yes” or “no” notifications to record any additional symptoms corresponding to the recorded disease. In one embodiment, the consolidated database is updated in real time based on the real time captured biophysical data.

The system 10 also includes a consent management module 50 operable by one or more processors. The consent management module 50 is operatively coupled to the to the health status recording module 40 and the biophysical data capturing module 30. The consent management module 50 is configured to classify the information received from the biophysical data capturing module 30 and the health status recording module 40 in accordance with predesignated privacy matrix level. In one embodiment, the predesignated privacy matrix level comprises criteria such as personal identifiable information, age, behavioural data and medical history, biological identifiers, lifestyle and education, types of diseases with privacy levels attributed to a specific disease and purpose of research.

In an exemplary embodiment, a patient X provides information for two diseases, first information regarding skin problem over face and second information regarding cataract problem in the left eye. As both require different specialization doctors, the consent management module 50 enable classifying the first and second information into different privacy matrix level. In another exemplary embodiment, a smart watch associated with the patient X records data regarding heart rate, blood pressure, calorie logging, calorie burning, body composition and the like. The consent management module 50 easily enables the patient X to classify each of such recorded information into different privacy matrix level. Further, the Doctors would also get to define access to the information generated during his/her treatment of the patient. In this case, the doctors would get to see the information as per the allowed privacy level access, hence complying with privacy requirement of the patient.

The consent management module 50 is also configured to provide access to a specific privacy level of classified information associated with a specific disease to one of one or more doctors, upon receiving consent from at least one of one or more registered doctors, the registered patient and the one or more registered healthcare personnel. In above stated both exemplary embodiments, it is vital to note that the patient X may according to need give access to classified matrix level information to each of the one or more registered doctors or each of the one or more healthcare personnel. In such embodiment, dissent provided by the registered patient supersedes over the consent of the one or more registered doctors.

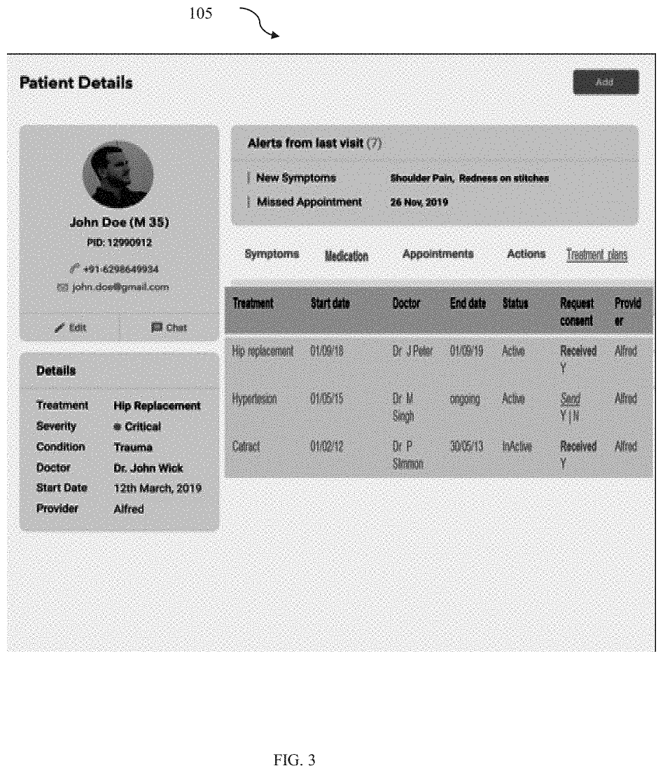

The system 10 also includes a graphic user interface module 60 operable by one or more processors. The graphic user interface module 60 is operatively coupled to the biophysical data capturing module 30, the health status recording module 40, and the consent management module 50. The graphic user interface module 60 is configured to provide integrated view of the information received from the biophysical data capturing module and the health status recording module to the one or more registered doctors treating the registered patient for one or more diseases based on the consent specific to privacy level of the classified information. Basically, the graphic user interface module 60 provides presentable view window module to the one or more registered doctors to understand various disease or aliments corresponding to a particular registered patient. FIG. 3 is a schematic representation of an embodiment representing the integrated presentation window 105 corresponding to the registered patient of FIG. 1 in accordance with an embodiment of the present disclosure.

In such embodiment, the graphic user interface module 60 is configured to prioritise intervention by the one or more registered doctors based on one of a seniority, specialisation, and disease being treated during the collaborative intervention. In one exemplary embodiment, a patient Z suffering from first stage lung cancer and COVID 19, will require monitoring by senior cancer specialist as well as and general physician. Intervention by cancer specialist is very much required as cancer is a life-threatening disease.

The graphic user interface module 60 is configured to enable the one or more registered doctors to collaboratively intervene according to information provided. In such embodiment, such collaboration may lead to creation of one or more treatment plans for real time implementation. Such treatment plans may be followed by the concerned registered patients. In such embodiment, the graphic user interface module 60 flags conflict during the collaboratively intervention by more than two doctors.

In above stated exemplary embodiment, the system 10 enables the senior cancer specialist as well as and general physician to collaborate for the patient Z. In such exemplary embodiment, if the patient Z is consulting any other doctor for any other illness or disorder, the system 10 flags any known conflict of recommended medicines. In an embodiment, the system 10 includes a database of the possible reactions of chemicals/medicines which is used as reference for flagging. In one such embodiment, the database is a combination of external data sources and internal data sources and intelligence that is developed overtime to identify conflict.

In another such embodiment, the system 10 will notify the doctor for reference while creating the treatment and will also send communications for the same in form of in-app notification. In yet another embodiment, the system 10 will ethically comply to policies mandated by Government of various regions globally. Here, the system 10 will configure and flag based on what is required as per the privacy laws and ethical use of medicine in a particular chosen region.

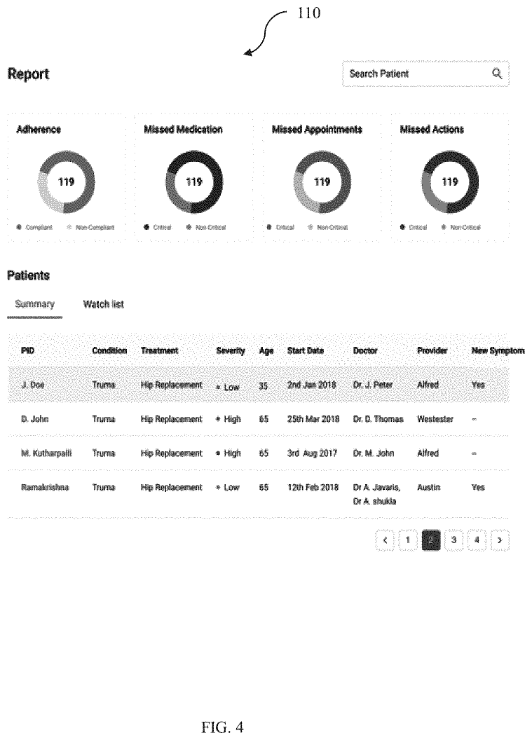

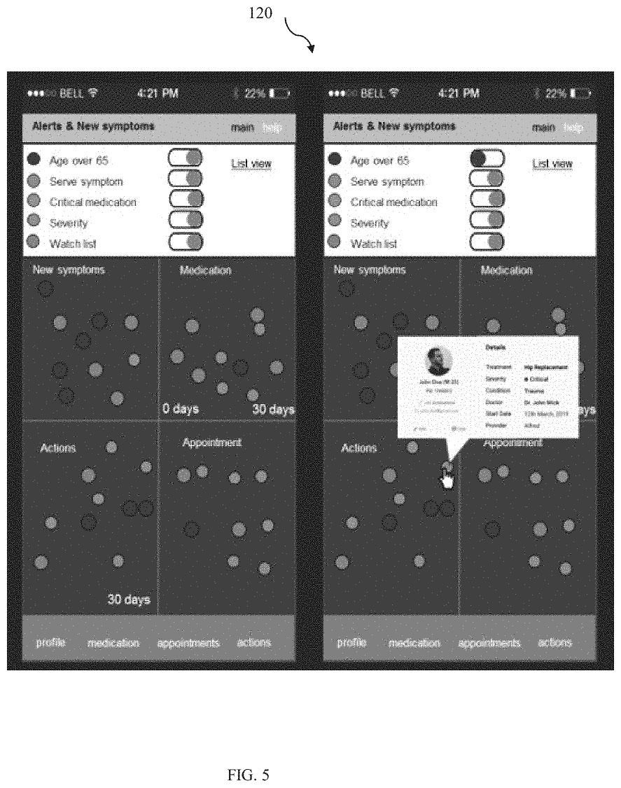

The graphic user interface module is configured to enforce adherence of the registered patient to the one or more treatment plans by processing the information received from the biophysical data capturing module and the health status recording module. FIG. 4 is a schematic representation of an embodiment representing the adherence dashboard window 110 corresponding to the registered patient of FIG. 1 in accordance with an embodiment of the present disclosure. FIG. 5 is a schematic representation of an embodiment representing the adherence dashboard window 120 with real time information corresponding to the registered patient of FIG. 1 in accordance with an embodiment of the present disclosure.

In one embodiment, the system 10 monitors adherences of the registered patient to the real-time generated treatment plan at a micro level. The adherence at the micro level comprises monitoring daily medication intakes, monitoring follow-up treatment compliance, monitoring medical appointments and monitoring ongoing symptoms and side-effects. The adherence dashboard window 110 & 120 provides details like missed appointment, missed actions, missed medication and the like. In such embodiment, the system in real time via notification gathers information about adherence of real time treatment plan. For example, the system might generate yes or no notification for intake of medicine at particular time of the day. Here, on reply of the notification from the registered patient, the system 10 might update the adherence dashboard window 110 & 120.

Also, the adherence report might be created daily or monthly to understand healthcare advancement of the registered patient. Upon non-adherence, the system 10 may be able to provide alert or notification to concerned patient contacts or healthcare personnel.

The system 10 also includes a feedback module operable by the one or more processors. The feedback module is operatively coupled to the graphic user interface module 60. The feedback module is configured to facilitate exchange of real-time feedbacks among the one or more registered entities, wherein the real-time feedbacks enable constant monitoring the registered patient health condition. Such real time feedbacks are vital for monitoring the registered patient health.

The system 10 also includes a health report generation module operable by the one or more processors. The health report generation module is operatively coupled to the graphic user interface module 60. The health report generation module is configured to generate a health report based on one or more enforced treatment plans.

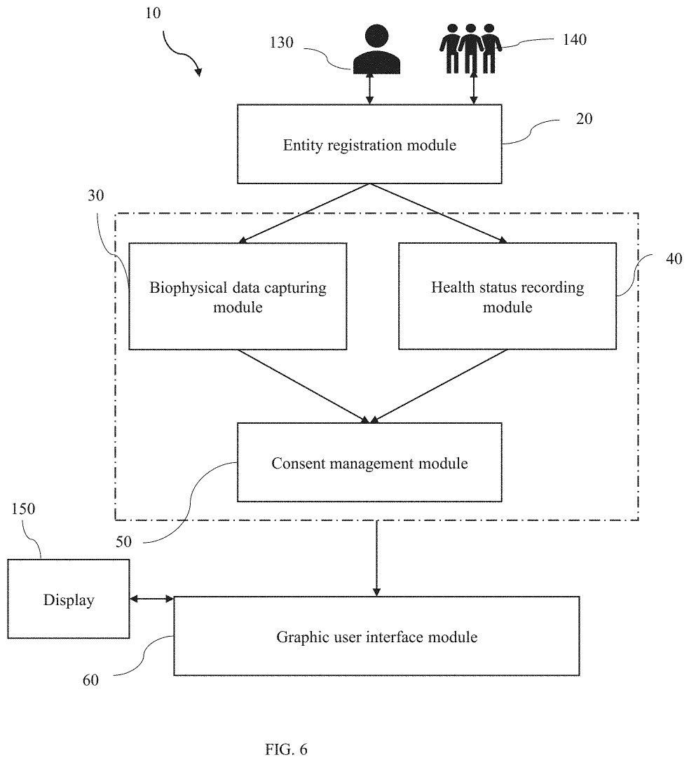

FIG. 6 is a schematic representation of an embodiment representing the system 10 to facilitate adherence to multiple medical care plans of FIG. 1 in accordance with an embodiment of the present disclosure. A patient M 130 at first instance registers into the system 10 via an entity registration module 20. During registration the patient M 130 provides details such as name, age medical history and the like. Likewise, one or more doctors 140 with different specialization from single hospital also registers into the system 10. During registration, each of the one or more doctors provides details such as specialization, years of experience and the like.

The patient M 130 via a biophysical data capturing module 30 adds disease symptoms and prior illness details. Here, through the biophysical data capturing module 30, the patient M 130 provide specifics of medical history related to abnormal blood pressure.

Further, the associated smart watch of the patient M 130 may also provide real time health details to the system 10 via a health status recording module 40. In the said exemplary embodiment, the patient M 130 shows new symptoms of heart problems associated with abnormal heart rate.

The patient M 130 may interact with a heart specialist 140 of the hospital via the system 10. Before interaction the patient M 130 may provide consent via a consent management module 50 on the privacy matric level about sharing medical history details regarding abnormal blood pressure. Such details are very much necessary for administrating medicine to the patient M 130. Furthermore, the heart specialist and the general physician 140 of the hospital may collaborate via a graphic user interface module 60 to help the patient M 130. Such collaboration may help in creating a common treatment plan without any medication conflict.

It is pertinent to note that the graphic user interface module 60 may also monitor adherence of the created treatment plan. In case of non-adherence, the system 10 may easily notify concerned person with proper alerts.

The entity registration module 20, the biophysical data capturing module 30, the health status recording module 40, the consent management module 50 and the graphic user interface module 60 in FIG. 6 is substantially equivalent to the entity registration module 20, the biophysical data capturing module 30, the health status recording module 40, the consent management module 50 and the graphic user interface module 60 of FIG. 1.

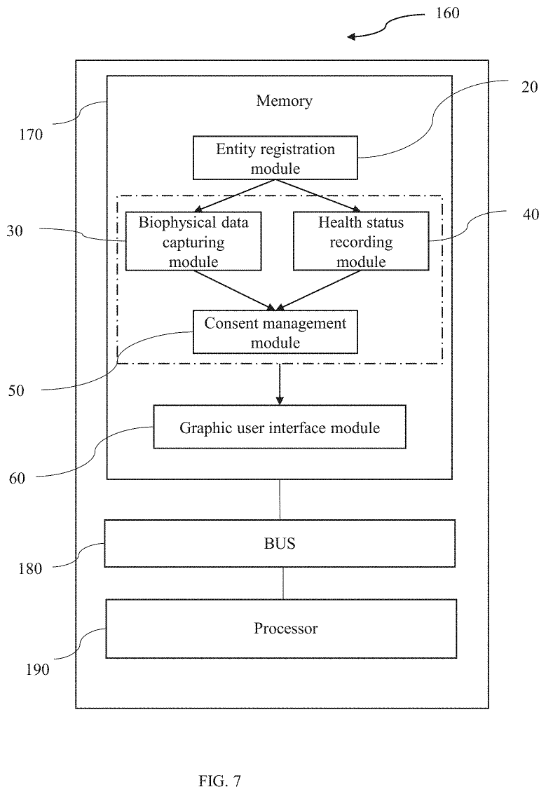

FIG. 7 is a block diagram of a computer or a server 160 in accordance with an embodiment of the present disclosure. The server 160 includes processor(s) 190, and memory 170 coupled to the processor(s) 190.

The processor(s) 190, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 170 includes a plurality of modules stored in the form of executable program which instructs the processor 190 via bus 180 to perform the method steps illustrated in FIG. 1. The memory 170 has following modules: entity registration module 20, the biophysical data capturing module 30, the health status recording module 40, the consent management module 50 and the graphic user interface module 60.

The entity registration module 20 is configured to register one or more entities with a plurality of information. The biophysical data capturing module 30 is configured to capture information regarding at least one of one or more diseases, one or more bio-physical parameters, type of discomfort, severity of discomfort, part of body experiencing the discomfort and time of discomfort, associated with the registered patient. The health status recording module 40 is configured to record medical information, in a consolidated database representative of one or more symptoms, one or more medication side-effects, ongoing medication, one or more treatment plans, and severity of health condition via communicatively coupled one or more devices associated with at least one of the registered patient, one or more registered healthcare personnel and one or more registered doctors.

The consent management module 50 is configured to classify the information received from the biophysical data capturing module and the health status recording module in accordance with predesignated privacy matrix level. The consent management module 50 is configured provide access to a specific privacy level of classified information associated with a specific disease to one of one or more doctors, upon receiving consent from at least one of one or more registered doctors, the registered patient and the one or more registered healthcare personnel.

The graphic user interface module 60 is configured to provide integrated view of the information received from the biophysical data capturing module and the health status recording module to the one or more registered doctors treating the registered patient for one or more diseases based on the consent specific to privacy level of the classified information. The graphic user interface module 60 is also configured to enable the one or more registered doctors to collaboratively intervene according to information provided, wherein the graphic user interface module flags conflict during the collaboratively intervention by more than two doctors. The graphic user interface module 60 is also configured to enforce adherence of the registered patient to the one or more treatment plans by processing the information received from the biophysical data capturing module and the health status recording module.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the processor(s) 190.

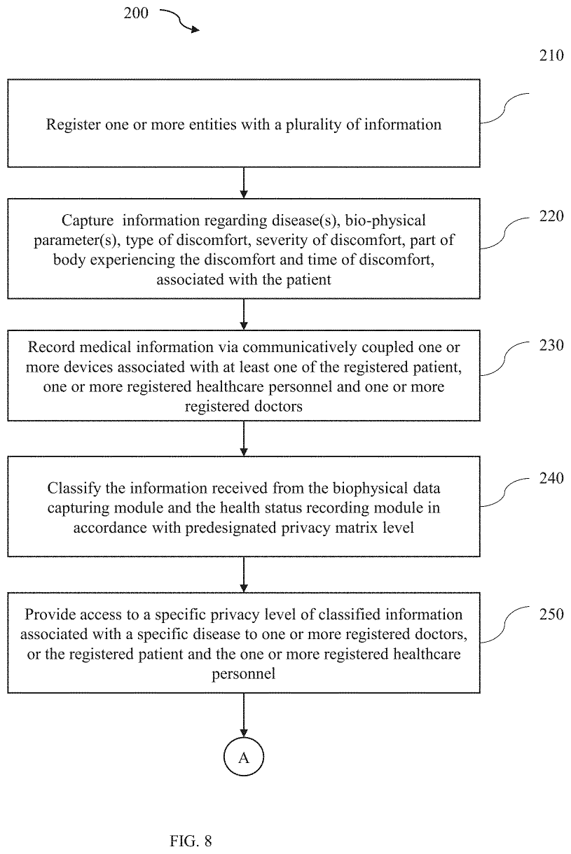

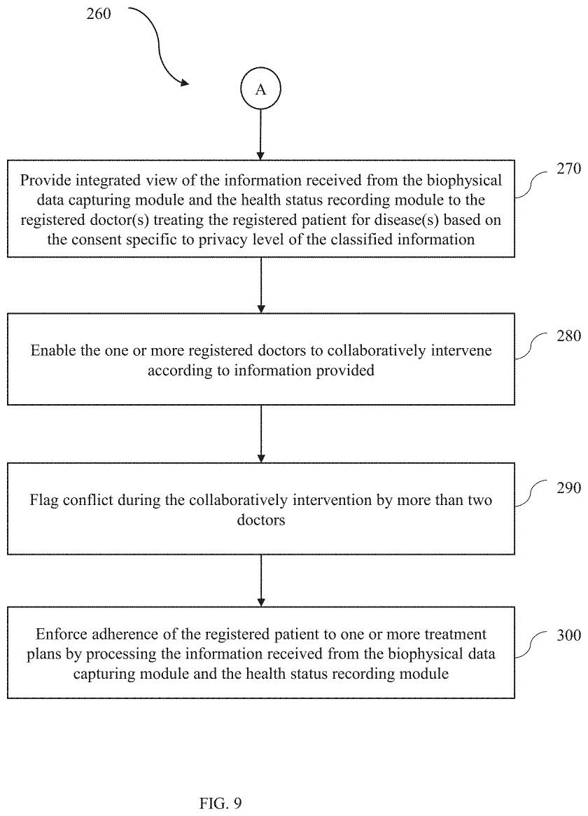

FIG. 8 is a flowchart representing the steps of a method 200 to facilitate adherence to multiple medical care plans in accordance with an embodiment of the present disclosure. FIG. 9 is a flowchart representing the steps of a method 260 to facilitate adherence to multiple medical care plans in accordance with an embodiment of the present disclosure.

The method 200 includes registering one or more entities with a plurality of information in step 210. In one embodiment, registering the one or more entities with the plurality of information includes registering the one or more entities with the plurality of information by an entity registration module. In one embodiment, registering the one or more entities with the plurality of information includes registering the one or more entities comprising a patient, one or more healthcare personnel, one or more doctors, an administrator and a patient contact, wherein the patient is undergoing treatment for one or more diseases by one or more doctors.

The method 200 also includes capturing information regarding at least one of one or more diseases, one or more bio-physical parameters, type of discomfort, severity of discomfort, part of body experiencing the discomfort and time of discomfort, associated with the patient in step 220. In one embodiment, capturing information regarding at least one of the one or more diseases, the one or more bio-physical parameters, the type of discomfort, the severity of discomfort, the part of body experiencing the discomfort and the time of discomfort associated with the patient includes capturing information by a biophysical data capturing module.

The method 200 also includes recording medical information representative of one or more symptoms, one or more medication side-effects, ongoing medication, one or more treatment plans, and severity of health condition via communicatively coupled one or more devices associated with at least one of the registered patient, one or more registered healthcare personnel and one or more registered doctors in step 230. In one embodiment, recording medical information representative of the one or more symptoms, the one or more medication side-effects, ongoing medication, the one or more treatment plans, and the severity of health condition via communicatively coupled the one or more devices associated with at least one of the registered patient, the one or more registered healthcare personnel and the one or more registered doctors includes recording medical information by a health status recording module.

The method 200 also includes classifying the information received from the biophysical data capturing module and the health status recording module in accordance with predesignated privacy matrix level in step 240. In one embodiment, classifying the information received from the biophysical data capturing module and the health status recording module in accordance with the predesignated privacy matrix level includes classifying the information received from the biophysical data capturing module and the health status recording module by a consent management module.

In one embodiment, classifying the information received from the biophysical data capturing module and the health status recording module in accordance with the predesignated privacy matrix level includes classifying in accordance with predesignated privacy matrix level comprising criteria such as personal identifiable information, age, behavioural data and medical history, biological identifiers, lifestyle and education, types of diseases with privacy levels attributed to a specific disease and purpose of research.

The method 200 also includes providing access to a specific privacy level of classified information associated with a specific disease to one of one or more registered doctors, upon receiving consent from at least one of one or more registered doctors, the registered patient and the one or more registered healthcare personnel in step 250. In one embodiment, providing access to the specific privacy level of classified the information associated with the specific disease to one of the one or more registered doctors, upon receiving consent from at least one of the one or more registered doctors, the registered patient and the one or more registered healthcare personnel includes providing access to a specific privacy level by the consent management module.

In another embodiment, providing access to the specific privacy level of classified the information associated with the specific disease to one of the one or more registered doctors, upon receiving consent from at least one of the one or more registered doctors, the registered patient and the one or more registered healthcare personnel includes providing non-access to the specific privacy level depending upon dissent received from the registered patient, wherein the registered patient consent supersedes over the consent of the one or more registered doctors.

The method 200 also includes providing integrated view of the information received from the biophysical data capturing module and the health status recording module to the one or more registered doctors treating the registered patient for one or more diseases based on the consent specific to privacy level of the classified information in step 270. In one embodiment, providing the integrated view of the information received from the biophysical data capturing module and the health status recording module to the one or more registered doctors treating the registered patient for one or more diseases based on the consent specific to privacy level of the classified information includes providing the integrated view of the information received from the biophysical data capturing module by a graphic user interface module.

The method 200 also includes enabling the one or more registered doctors to collaboratively intervene according to information provided in step 280. In one embodiment, enabling the one or more registered doctors to collaboratively intervene according to information includes enabling the one or more registered doctors to collaboratively intervene by the graphic user interface module.

The method 200 also includes prioritizing intervention by the one or more registered doctors based on one of a seniority, specialisation, and disease being treated during the collaborative intervention. In one embodiment, prioritizing intervention by the one or more registered doctors based on one of the seniorities, the specialisation, and the disease being treated during the collaborative intervention includes prioritizing intervention by the graphic user interface module.

The method 200 also includes flagging conflict during the collaboratively intervention by more than two doctors in step 290. In one embodiment, flagging conflict during the collaboratively intervention by more than two doctors includes flagging conflict during the collaboratively intervention by more than two doctors by the graphic user interface module.

The method 200 also includes enforcing adherence of the registered patient to the one or more treatment plans by processing the information received from the biophysical data capturing module and the health status recording module in step 300. In one embodiment, enforcing adherence of the registered patient to the one or more treatment plans by processing the information received from the biophysical data capturing module and the health status recording module includes enforcing adherence of the registered patient to the one or more treatment plans by the graphic user interface module.

The method 200 also includes monitoring adherence of the registered patient to the real-time generated treatment plan at a micro level. In one embodiment, monitoring adherence of the registered patient to the real-time generated treatment plan at the micro level includes monitoring by the graphic user interface module. In another embodiment, monitoring adherence of the registered patient to the real-time generated treatment plan at the micro level includes monitoring adherence of the registered patient to the real-time generated treatment plan at a micro level comprises monitoring daily medication intakes, monitoring follow-up treatment compliance, monitoring medical appointments and monitoring ongoing symptoms and side-effects.

The method 200 also includes comprising facilitating exchange of real-time feedbacks among the one or more registered entities, wherein the real-time feedbacks enable constant monitoring the registered patient health condition. In one embodiment, facilitating exchange of real-time feedbacks among the one or more registered entities, wherein the real-time feedbacks enable constant monitoring the registered patient health condition includes facilitating exchange of real-time feedbacks by a feedback module.

The method also includes comprising generating a health report based on one or more enforced treatment plans. In one embodiment, generating a health report based on one or more enforced treatment plans includes generating a health report based by a health report generation module.

Present disclosure of a system to facilitate adherence to multiple medical care plans provides a SaaS platform for real time collaboration of multiple doctors treating the same patient. The system, at first allows easy recording and capturing of medical information representative of one or more symptoms, one or more medication side-effects, ongoing medication, one or more treatment plans in a consolidated database. The system via a graphic user interface provides integrated view of treatments and symptoms to optimize and personalize care plans.

Further, the system facilitates easy consent management of such recorded information. Based on consent management policies what information is displayed to the doctors regarding other treatments from various other doctors will depend on regulations. Any patient may choose to disclose information about specific illness to his or her doctor. Moreover, each doctor may also choose to give consent of sharing treatment plans of a corresponding disease to other doctors. Hence, it ultimately helps in securely manage privacy.

The system additionally enables easy collaboration facility for doctors of multiple specialities to help treating a specific illness. The system also helps the patients to comply and adhere to the multiple treatment plans with extra monitoring. The system altogether enables a common standardisation and evolution of care.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependant on other acts may be performed in parallel with the other acts. The scope of embodiments is by no means limited by these specific examples.

We claim:

1. A system to facilitate adherence to multiple medical care plans, the system comprising:
one or more processors;
an entity registration module operable by the one or more processors, wherein the entity registration module is configured to register one or more entities with a plurality of information, wherein the one or more entities comprises a patient, one or more healthcare personnel, one or more doctors, an administrator and a patient contact, wherein the patient is undergoing treatment for one or more diseases by one or more doctors;
a biophysical data capturing module operable by the one or more processors and operatively coupled to the entry registration module, wherein the biophysical data capturing module is configured to
capture information regarding at least one of one or more diseases, one or more bio-physical parameters, type of discomfort, severity of discomfort, part of body experiencing the discomfort and time of discomfort, associated with the registered patient;
a health status recording module operable by the one or more processors, and operatively coupled to the entity registration module, wherein the health status recording module is configured to
record medical information, in a consolidated database representative of one or more symptoms, one or more medication side-effects, ongoing medication, one or more treatment plans, and severity of health condition via communicatively coupled one or more devices associated with at least one of the registered patient, one or more registered healthcare personnel and one or more registered doctors;
a consent management module operable by one or more processors, and operatively coupled to the to the health status recording module and the biophysical data capturing module, wherein the consent management module is configured to:
classify the information received from the biophysical data capturing module and the health status recording module in accordance with predesignated privacy matrix level; and
provide access to a specific privacy level of classified information associated with a specific disease to one of one or more doctors, upon receiving consent from at least one of one or more registered doctors, the registered patient and the one or more registered healthcare personnel, wherein dissent provided by the registered patient supersedes over the consent of the one or more registered doctors;
a graphic user interface module operable by one or more processors, and operatively coupled to the biophysical data capturing module, the health status recording module, and the consent management module, wherein the graphic user interface module is configured to:
provide integrated view of the information received from the biophysical data capturing module and the health status recording module to the one or more registered doctors treating the registered patient for one or more diseases based on the consent specific to privacy level of the classified information;
enable the one or more registered doctors to collaboratively intervene according to information provided, wherein the graphic user interface module flags conflict during the collaboratively intervention by more than two doctors; and
enforce adherence of the registered patient to the one or more treatment plans by processing the information received from the biophysical data capturing module and the health status recording module.

2. The system as claimed in claim 1, wherein the graphic user interface module is configured to prioritise intervention by the one or more registered doctors based on one of a seniority, specialisation, and disease being treated during the collaborative intervention.

3. The system as claimed in claim 1, wherein the system is hosted on a Software as a service (SaaS) platform.

4. The system as claimed in claim 1, wherein the predesignated privacy matrix level comprises criteria such as personal identifiable information, age, behavioural data and medical history, biological identifiers, lifestyle and education, types of diseases with privacy levels attributed to a specific disease and purpose of research.

5. The system as claimed in claim 1, wherein monitor adherence of the registered patient to the real-time generated treatment plan at a micro level, wherein the adherence at the micro level comprises monitoring daily medication intakes, monitoring follow-up treatment compliance, monitoring medical appointments and monitoring ongoing symptoms and side-effects.

6. The system as claimed in claim 1, comprising a feedback module operable by the one or more processors and operatively coupled to the graphic user interface module, wherein the feedback module is configured to facilitate exchange of real-time feedbacks among the one or more registered entities, wherein the real-time feedbacks enable constant monitoring the registered patient health condition.

7. The system as claimed in claim 1, comprising a health report generation module operable by the one or more processors and operatively coupled to the graphic user interface module, wherein the health report generation module is configured to generate a health report based on one or more enforced treatment plans.

8. A method for facilitating adherence to multiple medical care plans, the method comprising:
registering, by an entity registration module, one or more entities with a plurality of information;
capturing, by a biophysical data capturing module, information regarding at least one of one or more diseases, one or more bio-physical parameters, type of discomfort, severity of discomfort, part of body experiencing the discomfort and time of discomfort, associated with the patient;
recording medical information, by a health status recording module, representative of one or more symptoms, one or more medication side-effects, ongoing medication, one or more treatment plans, and severity of health condition via communicatively coupled one or more devices associated with at least one of the registered patient, one or more registered healthcare personnel and one or more registered doctors;
classifying, by a consent management module, the information received from the biophysical data capturing module and the health status recording module in accordance with predesignated privacy matrix level;
providing, by the consent management module, access to a specific privacy level of classified information associated with a specific disease to one of one or more registered doctors, upon receiving consent from at least one of one or more registered doctors, the registered patient and the one or more registered healthcare personnel;
providing, by a graphic user interface module, integrated view of the information received from the biophysical data capturing module and the health status recording module to the one or more registered doctors treating the registered patient for one or more diseases based on the consent specific to privacy level of the classified information;
enabling, by the graphic user interface module, the one or more registered doctors to collaboratively intervene according to information provided;
flagging, by the graphic user interface module, conflict during the collaboratively intervention by more than two doctors; and
enforcing, by the graphic user interface module, adherence of the registered patient to one or more treatment plans by processing the information received from the biophysical data capturing module and the health status recording module.

9. The method as claimed in claim 8, registering, by an entity registration module, one or more entities comprising a patient, one or more healthcare personnel, one or more doctors, an administrator and a patient contact, wherein the patient is undergoing treatment for one or more diseases by one or more doctors.

10. The method as claimed in claim 8, wherein providing, by the consent management module, non-access to the specific privacy level depending upon dissent received from the registered patient, wherein the registered patient consent supersedes over the consent of the one or more registered doctors.

11. The method as claimed in claim 8, comprising prioritizing, by the graphic user interface module, intervention by the one or more registered doctors based on one of a seniority, specialisation, and disease being treated during the collaborative intervention.

12. The method as claimed in claim 8, wherein classifying, by a consent management module, in accordance with predesignated privacy matrix level comprising criteria such as personal identifiable information, age, behavioural data and medical history, biological identifiers, lifestyle and education, types of diseases with privacy levels attributed to a specific disease and purpose of research.

13. The method as claimed in claim 8, comprising monitoring, by the graphic user interface module, adherence of the registered patient to the real-time generated treatment plan at a micro level.

14. The method as claimed in claim 13, monitoring, by the graphic user interface module, adherence of the registered patient to the real-time generated treatment plan at a micro level comprises monitoring daily medication intakes, monitoring follow-up treatment compliance, monitoring medical appointments and monitoring ongoing symptoms and side-effects.

15. The method as claimed in claim 8, comprising facilitating, by a feedback module, exchange of real-time feedbacks among the one or more registered entities, wherein the real-time feedbacks enable constant monitoring the registered patient health condition.

16. The method as claimed in claim 6, comprising generating, by a health report generation module, a health report based on one or more enforced treatment plans.
